(19) United States
(12) Patent Application Publication
(10) Pub. No.: US 2026/0115461 A1
(43) Pub. Date: Apr. 30, 2026

(54) CONNECTION QUALITY DETERMINATION FOR WEARABLE NEUROSTIMULATION SYSTEMS

(71) Applicant: Cala Health, Inc., San Mateo, CA (US)

(72) Inventors: Sami Balbaky, San Jose, CA (US); Samuel Richard Hamner, San Francisco, CA (US)

(21) Appl. No.: 19/030,173

(22) Filed: Jan. 17, 2025

Related U.S. Application Data
(63) Continuation of application No. 16/993,085, filed on Aug. 13, 2020, now Pat. No. 12,251,560.
(60) Provisional application No. 62/886,218, filed on Aug. 13, 2019.

(51) Int. Cl. A61N 1/08; A61N 1/36

(52) U.S. Cl. CPC A61N 1/08; A61N 1/36142; A61N 1/36175; A61N 2001/083

(57) ABSTRACT

Systems, devices, and methods for electrically stimulating peripheral nerve(s) to treat various disorders are disclosed, as well as signal processing systems and methods for enhancing diagnostic and therapeutic protocols relating to the same.

### REFERENCE TO RELATED APPLICATIONS

This application is a continuation of U.S. application Ser. No. 16/993,085, filed on Aug. 13, 2020, which claims the benefit under 35 U.S.C. § 119(e) to U.S. Prov. App. No. 62/886,218 filed on Aug. 13, 2019, each of which is hereby incorporated by reference in its entirety.

### BACKGROUND

### Field

Embodiments of the invention relate generally to systems, devices, and methods for stimulating nerves, and more specifically relate to system, devices, and methods for electrically stimulating peripheral nerve(s) to treat various disorders, as well as signal processing systems and methods for enhancing diagnostic and therapeutic protocols relating to the same.

### Description of the Related Art

A wide variety of modalities can be utilized to neuromodulate peripheral nerves. For example, electrical energy can be delivered transcutaneously or percutaneously via electrodes on the skin surface with neurostimulation systems to stimulate peripheral nerves, such as the median, radial, and/or ulnar nerves in the upper extremities; the tibial, saphenous, and/or peroneal nerve in the lower extremities; or the auricular vagus, tragus, trigeminal, occipital, or cranial nerves on the head or ear, as non-limiting examples. Stimulation of these nerves has been shown to provide therapeutic benefit across a variety of diseases, including but not limited to movement disorders (including but not limited to essential tremor, Parkinson's tremor, orthostatic tremor, and multiple sclerosis), urological disorders, gastrointestinal disorders, cardiac diseases, and inflammatory diseases, mood disorders (including but not limited to depression, bipolar disorder, dysthymia, and anxiety disorder), pain syndromes (including but not limited to migraines and other headaches, trigeminal neuralgia, fibromyalgia, complex regional pain syndrome), among others. A number of conditions, such as tremors, can be treated through some form of transcutaneous, percutaneous, or other implanted forms of peripheral nerve stimulation.

### SUMMARY

Systems with compact, ergonomic form factors are needed to enhance efficacy, compliance, and/or comfort when using non-invasive or wearable neuromodulation devices. In several embodiments, neuromodulation systems and methods are provided that enhance or inhibit nerve impulses and/or neurotransmission, and/or modulate excitability of nerves, neurons, neural circuitry, and/or other neuroanatomy that affects activation of nerves and/or neurons. For example, neuromodulation (e.g., neurostimulation) can include one or more of the following effects on neural tissue: depolarizing the neurons such that the neurons fire action potentials; hyperpolarizing the neurons to inhibit action potentials; depleting neuron ion stores to inhibit firing action potentials; altering with proprioceptive input; influencing muscle contractions; affecting changes in neurotransmitter release or uptake; and/or inhibiting firing.

In some embodiments, disclosed here is a wearable neurostimulation device for transcutaneously stimulating one or more peripheral nerves of a user. The device can include, for example, any number of a plurality of electrodes configured to generate electric stimulation signals; and one or more hardware processors configured to: measure a plurality of distinct electromagnetic characteristics across the at least two electrodes; determine a plurality of connection quality indicators based on the measured plurality of electromagnetic characteristics, said connection quality indicators corresponding to a degree of connection between the at least two of the plurality of electrodes and skin of the user; update the plurality of connection quality indicators over a time window based on repeated measurements of the plurality of electromagnetic characteristics within the time window; determine that at least one of the plurality of connection quality indicators crosses a threshold within the time window; and/or change an operating characteristic of the wearable neurostimulation device based on the determination that the at least one of the plurality of connection quality indicators has crossed the threshold within the time window.

In some embodiments, the one or more hardware processors are configured to reset the plurality of connection quality indicators for a new time window.

In some embodiments, the one or more hardware processors are configured to maintain therapy during the time window where at least one of the plurality of connection quality indicators indicate a weak connection and where at least one of the plurality of connection quality indicators has not crossed the threshold.

In some embodiments, the electromagnetic characteristic comprises impedance.

In some embodiments, electromagnetic characteristic comprises voltage mismatch.

In some embodiments, the electromagnetic characteristic comprises a pulse shape In some embodiments, the operating characteristic includes stimulation settings.

In some embodiments, changing the operating characteristic includes generating an alert.

In some embodiments, each of the plurality of electromagnetic characteristics is configured to enable an independent determination of the connection quality indicator.

In some embodiments, disclosed herein is a method for monitoring the connection quality of a neurostimulation device. The method can include, for example, any number of the following: measuring a plurality of distinct electromagnetic characteristics across the at least two electrodes; determining a plurality of connection quality indicators based on the measured plurality of electromagnetic characteristics, said connection quality indicators corresponding to a degree of connection between the at least two of the plurality of electrodes and skin of the user; updating the plurality of connection quality indicators over a time window based on repeated measurements of the plurality of electromagnetic characteristics within the time window; determining that at least one of the plurality of connection quality indicators crosses a threshold within the time window; and changing an operating characteristic of the wearable neurostimulation device based on the determination that the at least one of the plurality of connection quality indicators has crossed the threshold within the time window.

In some embodiments, the method includes resetting the plurality of connection quality indicators for a new time window.

In some embodiments, the method includes maintaining therapy during the time window where at least one of the plurality of connection quality indicators indicate a weak connection and where at least one of the plurality of connection quality indicators has not crossed the threshold.

In some embodiments, the electromagnetic characteristic comprises impedance.

In some embodiments, the electromagnetic characteristic comprises voltage mismatch.

In some embodiments, the electromagnetic characteristic comprises a pulse shape determination.

In some embodiments, the operating characteristic includes stimulation settings.

In some embodiments, the changing the operating characteristic includes generating an alert.

In some embodiments, each of the plurality of electromagnetic characteristics is configured to enable an independent determination of the connection quality indicator.

In some embodiments, also disclosed herein is a wearable neurostimulation device for transcutaneously stimulating one or more peripheral nerves of a user. The device can include, for example, any number of the following: a plurality of electrodes configured to generate electric stimulation signals; and one or more hardware processors configured to: determine an electromagnetic characteristic across at least two of the plurality of electrodes; determine a connection quality indicator, corresponding to connection between the at least two of the plurality of electrodes and skin of the user, based on the determined electromagnetic characteristic; and/or change an operating characteristic of the wearable neurostimulation device based on the determined connection quality indicator.

In some embodiments, the electromagnetic characteristic comprises impedance.

In some embodiments, the electromagnetic characteristic comprises voltage mismatch.

In some embodiments, the electromagnetic characteristic comprises a pulse shape determination.

In some embodiments, the operating characteristic includes stimulation settings.

In some embodiments, the changing the operating characteristic includes generating an alert.

In some embodiments, the one or more hardware processors is configured to determine a plurality of electromagnetic characteristics across the at least two electrodes.

In some embodiments, each of the plurality of electromagnetic characteristics is configured to enable an independent determination of the connection quality indicator.

In some embodiments, the one or more hardware processors is configured to determine a plurality of connection quality indicators based on the determined plurality of electromagnetic characteristics.

In some embodiments, the one or more hardware processors is configured to track the determined plurality of connection quality indicators over a time period.

In some embodiments, the operating characteristic is only changed when at least one of the determined plurality of connection quality indicators exceed a threshold over the time period.

In some embodiments, disclosed herein is a wearable neurostimulation device for transcutaneously stimulating one or more peripheral nerves of a user. The device can include, for example, any number of the following: a plurality of electrodes configured to generate electric stimulation signals; and one or more hardware processors configured to: determine a plurality of electromagnetic characteristics across at least two of the plurality of electrodes; determine a plurality of connection quality indicators, corresponding to connection between the at least two of the plurality of electrodes and skin of the user, from each of the determined electromagnetic characteristic; track the plurality of connection quality indicators over a time window; and/or change an operating characteristic of the wearable neurostimulation device based on the tracking of the plurality of connection quality indicators.

In some embodiments, the plurality of electrodes comprise dry electrodes.

In some embodiments, the plurality of electrodes comprise a hydrogel.

In some embodiments, the plurality of electromagnetic characteristics comprise one or more of: impedance, voltage mismatch, and pulse shape determination.

In some embodiments, the one or more hardware processors is configured to track the plurality of connection quality indicators over a time window by maintaining one or more counters.

In some embodiments, the one or more hardware processors is configured to track the plurality of connection quality indicators over a time window by maintaining only a single counter based on the plurality of connection quality indicators.

In some embodiments, the one or more hardware processors is configured to change the operating characteristic of the wearable neurostimulation device if the plurality of connection quality indicators exceeds a threshold over the time window.

In some embodiments, the one or more hardware processors is configured to weight the plurality of connection quality indicators.

In some embodiments, the one or more hardware processors is configured to generate an alert to the patient.

In some embodiments, the one or more hardware processors is configured to change the operating characteristic of the wearable neurostimulation device by discontinuing stimulation.

In some embodiments, disclosed herein is a method for monitoring the connection quality of a neurostimulation device, that can include, for example, any number of the following: positioning a plurality of electrodes of the neurostimulation device on a skin of a user; delivering electrical stimulation transcutaneously through the plurality of electrodes; determining a plurality of electromagnetic characteristics across at least two of the plurality of electrodes; determining a plurality of connection quality indicators, corresponding to connection between the at least two of the plurality of electrodes and the skin of the user; tracking the plurality of connection quality indicators over a time window; and/or changing an operating characteristic of the wearable neurostimulation device if the plurality of connection quality indicators exceeds a threshold over the time window.

In some embodiments, the method includes determining a plurality of electromagnetic characteristics comprises determining one or more of impedance, voltage mismatch, and pulse shape.

In some embodiments, tracking the plurality of connection quality indicators comprises maintaining at least one counter of connection quality over the time window based on the plurality of connection quality indicators.

In some embodiments, tracking the plurality of connection quality indicators comprises maintaining only a single counter of connection quality over the time window based on the plurality of connection quality indicators.

In some embodiments, the method includes initializing a value of the counter after the end of the time window.

In some embodiments, the method includes altering a value of the counter based on at least one of the plurality of connection quality indicators.

In some embodiments, changing the operating characteristic comprises discontinuing delivering the electrical stimulation, and/or modifying the delivered electrical stimulation.

In some embodiments, modifying the delivered electrical stimulation comprises changing one or more of the frequency, voltage, current, or duration of the stimulation.

In some embodiments, the method also includes alerting the user to an abnormality in the connection quality.

In some embodiments, disclosed herein is a wearable neuromodulation device for transcutaneously modulating one or more peripheral nerves of a user. The device can include, for example, any number of the following: a plurality of effectors configured to generate neuromodulation signals; and one or more hardware processors configured to: determine a characteristic across at least two of the plurality of effectors; determine a connection quality indicator, corresponding to connection between the at least two of the plurality of effectors and skin of the user, based on the determined characteristic; and/or change an operating characteristic of the wearable neuromodulation device based on the determined connection quality indicator.

In some embodiments, disclosed herein is a wearable neuromodulation device for transcutaneously modulating one or more peripheral nerves of a user. The device can include, for example, any number of the following: a plurality of effectors configured to generate neuromodulation signals; and one or more hardware processors configured to: determine a plurality of characteristic across at least two of the plurality of effectors; determine a plurality of connection quality indicators, corresponding to connection between the at least two of the plurality of effectors and skin of the user, from each of the determined electromagnetic characteristic; track the plurality of connection quality indicators over a time window; and/or change an operating characteristic of the wearable neuromodulation device based on the tracking of the plurality of connection quality indicators.

In some embodiments, disclosed herein is a wearable neuromodulation device for transcutaneously modulating one or more peripheral nerves of a user. The device can include, for example, any number of the following: a plurality of electrodes configured to generate electric stimulation signals; and one or more hardware processors configured to: measure one, two, three, four, five, or more distinct electromagnetic characteristics across the at least two electrodes; determine one, two, three, four, five, or more connection quality indicators based on the measured one or more electromagnetic characteristics, said connection quality indicators corresponding to a degree of connection between the at least two, three, four, five, or more of the plurality of electrodes and skin of the user; update the one, two, or more connection quality indicators over a time window based on repeated measurements of the one, two, three, four, five, or more electromagnetic characteristics within the time window; determine that one, two, three, four, five, or more of the connection quality indicators crosses a threshold within the time window; and/or change an operating characteristic of the wearable neurostimulation device based on the determination that the one, two, three, four, five, or more of the connection quality indicators has crossed the threshold within the time window.

In some embodiments, disclosed herein is a neuromodulation device according to any one of the embodiments described in the disclosure. A device can, in some embodiments, comprise, consist essentially of, consist of, and/or not comprise any number of features as disclosed herein.

In some embodiments, disclosed herein is a method for tracking one or more connection quality indicators according to any one of the embodiments described in the disclosure.

In some embodiments, disclosed herein is a method for changing an operating characteristic of a wearable neuromodulation device based on tracking of a plurality of connection quality indicators.

In several embodiments, the embodiments described herein that, for example, monitor the connection quality of a neuromodulation system can have one or more of the following advantages: (i) greater therapeutic benefit with less discomfort (e.g., such as from electrical discharge from open circuits); (ii) less current use (e.g., less power and improved battery life); (iii) decreased device error alerts and interruptions in therapy (and thus delays in completing a therapy session); (iv) increased likelihood of patient compliance due to the foregoing; and/or (v) improving the sensitivity in the determination of when to generate the alert and/or stop delivery of the electrical stimulation to balance the safety of users with efficacy and comfort.

In some of the embodiments described herein, one, several or all of the following features are not included: (i) sensors configured to assess patient motion and/or collect motion data, (ii) accelerometers, gyroscopes, magnetometers, inertial measurement units. and (iii) EMG or other muscle sensors. In some embodiments, systems and methods are not configured for, or are not placed on the upper arm and/or are not configured for neuromodulation on the skin surface of the forehead. In some embodiments, systems and methods are not configured to, or do not modulate descending (e.g., efferent) nerve pathways, and only modulate ascending (e.g., afferent) nerve pathways. In some embodiments, systems and methods are not configured to, or do not modulate nerves only on the ventral side of the wrist. In some embodiments, systems and methods do not include any implantable components. In some embodiments, systems and methods are not configured for percutaneous or subcutaneous stimulation, and are only configured for transcutaneous neuromodulation. In some embodiments, systems and methods are not configured for only neuromodulating, e.g., stimulating the ventral side of the wrist, rather some configurations may neuromodulate, e.g., deliver stimulation between two or more of the ventral, dorsal, and/or lateral sides of the wrist to target the medial nerve.

### BRIEF DESCRIPTION OF THE DRAWINGS

FIG. 1A illustrates a block diagram of an example neuromodulation (e.g., neurostimulation) device.

FIG. 1B illustrates a block diagram of an embodiment of a controller that can be implemented with the hardware components described with respect to FIG. 1A.

FIG. 1C illustrates a block diagram of an embodiment of a controller.

FIG. 2 illustrates a flow chart of an embodiment of a process for determination of connection quality using impedance determination.

FIG. 3 illustrates a flow chart of an embodiment of a process for determination of connection quality using pulse shape determination.

FIG. 4 illustrates a flow chart of an embodiment of a process for determination of connection quality using voltage mismatch.

FIG. 5A illustrates a flow chart of an embodiment of a process for determination of connection quality.

FIG. 5B illustrates an architecture for determination of connection quality using more than one of the processes over time.

FIG. 5C illustrates a flow chart of an embodiment of a process for adding a time component to the processes discussed above.

### DETAILED DESCRIPTION

Several embodiments disclosed herein relate to systems and methods for neuromodulation, including for example devices configured for providing neurostimulation. The neuromodulation (e.g., neurostimulation) devices provided herein may be configured to stimulate peripheral nerves of a user. The devices may be configured to transcutaneously transmit one or more neuromodulation (e.g., neurostimulation) signals across the skin of the user. In many embodiments, the devices are wearable devices configured to be worn by a user. The user may be a human, another mammal, or other animal user. A neuromodulation (e.g., neurostimulation) system is also provided in several embodiments and includes signal processing systems and methods for enhancing diagnostic and therapeutic protocols relating to the same. In some embodiments, the neuromodulation (e.g., neurostimulation) device is configured to be wearable on an upper extremity of a user (e.g., a wrist, forearm, arm, and/or finger(s) of a user). In some embodiments, the device is configured to be wearable on a lower extremity (e.g., ankle, calf, knee, thigh, foot, and/or toes) of a user. In some embodiments, the device is configured to be wearable on the head or neck (e.g., forehead, ear, neck, nose, and/or tongue). In several embodiments, dampening or blocking of nerve impulses and/or neurotransmitters are provided. In some embodiments, nerve impulses and/or neurotransmitters are enhanced.

In some embodiments, the device is configured to be wearable on or proximate an ear of a user, including but not limited to auricular neuromodulation (e.g., neurostimulation) of the auricular branch of the vagus nerve, for example. The device could be unilateral or bilateral, including a single device or multiple devices connected with wires or wirelessly.

In some embodiments, the neuromodulation (e.g., neurostimulation) devices and methods disclosed herein generally rely on a robust connection with the skin of the user. In some embodiments, the electrode material conforms to the irregularities on the skin surface to ensure a robust and proper electrical connection. Conformance of the electrode to the skin surface can be affected by properties of the electrode, including but not limited to shape, thickness, material compliance (e.g., stiffness or durometer), and/or adhesion properties. Particularly, due to an application of electrical stimulation, an improper connection can result, for example, in current concentration that may make the device inoperable and/or may be harmful or painful to the user. For example, current concentrations can occur as an electrode lifts from the skin surface thus reducing the area of contact and, if current delivered by the device is held constant, increasing the current density. The connection quality may vary based on user movement. The connection state or quality between the skin and the effectors, e.g., electrode(s) may also depend on the type material used for the electrodes. In some instances, to improve the quality of user experience, especially for all day wear or multiple reapplications, the effectors, e.g., electrodes, may not include an adhesive hydrogel material. While an adhesive hydrogel layer can improve conformance with the skin, the stickiness of the hydrogel can be uncomfortable for the user or collect dust and debris during wear. This can sometimes increase the risk of poor connection quality. In some embodiments, the wearer is instructed to wet the wrist or electrodes with water, gel, lotion, or another conductive medium to improve the connection at the skin interface. Without a proper connection between the neuromodulation device and the user, the overall efficacy can be reduced in some embodiments due to improper delivery of electrical stimulation to the appropriate nerve targets or reduced adherence with the recommended usage due to discomfort or device errors (e.g., device not operating normally). Accordingly, some of the systems and methods described herein improve the efficacy of treatment by determining the connection quality between the device and the user. It has been observed that an increase in device errors may cause some users to not comply with their therapy regime due to either frustration with the device or possible reduction in therapeutic effect. Accordingly, in some embodiments, increased connection quality results in enhanced comfort for the user and reduce number of device disconnection errors, thus increasing compliance or adherence.

When the quality of the connection between the neuromodulation (e.g., neurostimulation) device and the user is poor, the device can be programmed to halt the stimulation to avoid improper delivery of electrical stimulation. The device can be further programmed to alert a user of the poor quality connection. However, there may be instances where false positives are generated. For example, connection may be poor only for a small time period while the user is moving and then stabilize. However, if alerts are constantly generated when the connection is poor, this may adversely affect user experience. Moreover, if the stimulation/modulation is halted every time when there is some indication of poor connection quality, it may reduce the efficacy of the treatment, and/or causes delays in completing a therapy session. Accordingly, the systems and methods described herein improve the sensitivity in the determination of when to generate the alert and/or stop delivery of the electrical stimulation to balance the safety of users with efficacy and comfort.

### Neuromodulation Device

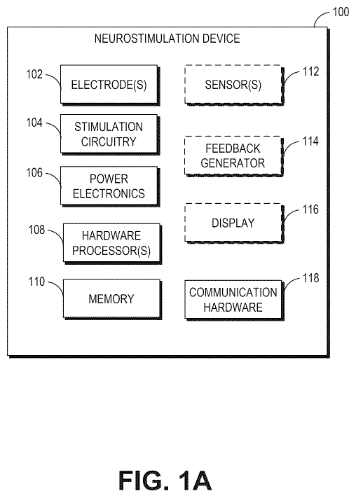

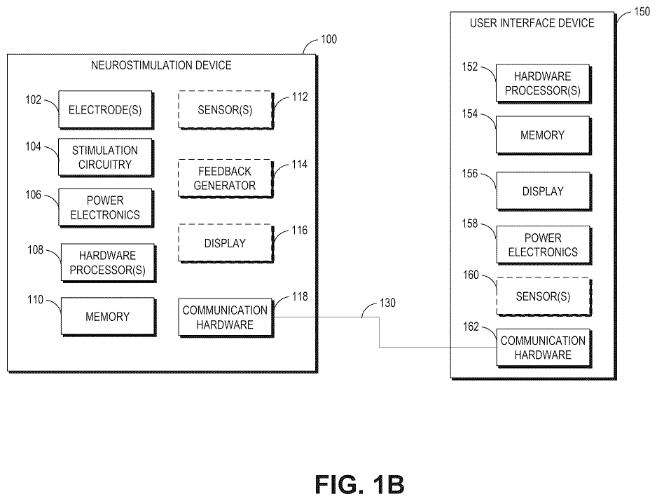

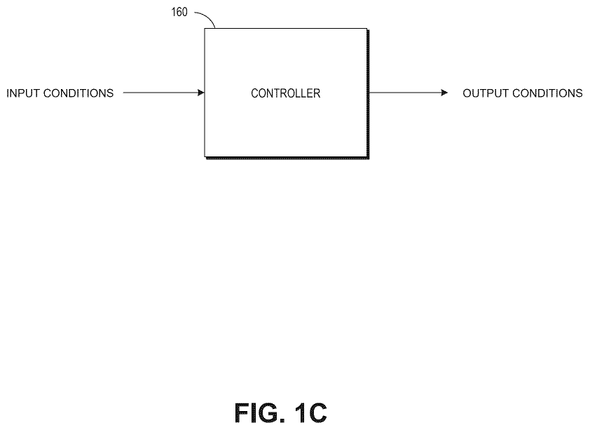

FIG. 1A illustrates a block diagram of an example neuromodulation (e.g., neurostimulation) device 100. The device 100 includes multiple hardware components which are capable of, or programmed to provide therapy across the skin of the user. As illustrated in FIG. 1A, some of these hardware components may be optional as indicated by dashed blocks. In some instances, the device 100 may only include the hardware components that are required for stimulation therapy. The hardware components are described in more detail below.

The device 100 can include two or more effectors, e.g. electrodes 102 for providing neurostimulation signals. In some instances, the device 100 is configured for transcutaneous use only and does not include any percutaneous or implantable components. In some embodiments, the electrodes can be dry electrodes. In some embodiments, water or gel can be applied to the dry electrode or skin to improve conductance. In some embodiments, the electrodes do not include any hydrogel material, adhesive, or the like. The electrodes 102 can also be used to determine a connection quality as discussed below.

The device 100 can further include stimulation circuitry 104 for generating signals that are applied through the electrode(s) 102. The signals can vary in frequency, phase, timing, amplitude, or offsets. The device 100 can also include power electronics 106 for providing power to the hardware components. For example, the power electronics 106 can include a battery.

The device 100 can include one or more hardware processors 108. The hardware processors 108 can include microcontrollers, digital signal processors, application specific integrated circuit (ASIC), a field programmable gate array (FPGA) or other programmable logic device, discrete gate or transistor logic, discrete hardware components, or any combination thereof designed to perform the functions described herein. In an embodiment, all of the processing discussed herein is performed by the hardware processor(s) 108. The memory 110 can store data specific to patient and rules as discussed below.

In the illustrated figure, the device 100 can include one or more sensors 112. As shown in the figure, the sensor(s) 112 may be optional. Sensors could include, for example, biomechanical sensors configured to, for example, measure motion, and/or bioelectrical sensors (e.g., EMG, EEG, and/or nerve conduction sensors). Sensors can include, for example, cardiac activity sensors (e.g., ECG, PPG), skin conductance sensors (e.g., galvanic skin response, electrodermal activity), and motion sensors (e.g., accelerometers, gyroscopes). The one or more sensors 102 may include an inertial measurement unit (IMU).

In some embodiments, the IMU can include one or more of a gyroscope, accelerometer, and magnetometer, which may also be used to determine connection quality. The IMU can be affixed or integrated with the neuromodulation (e.g., neurostimulation) device 100. In an embodiment, the IMU is an off the shelf component. In addition to its ordinary meaning, the IMU can also include specific components as discussed below. For example, the IMU can include one more sensors capable of collecting motion data. In an embodiment, the IMU includes an accelerometer. In some embodiments, the IMU can include multiple accelerometers to determine motion in multiple axes. Furthermore, the IMU can also include one or more gyroscopes and/or magnetometer in additional embodiments. Since the IMU can be integrated with the neurostimulation device 100, the IMU can generate data from its sensors responsive to motion, movement, or vibration felt by the device 100. Furthermore, when the device 100 with the integrated IMU is worn by a user, the IMU can enable detection of voluntary and/or involuntary motion of the user.

The device 100 can optionally include user interface components, such as a feedback generator 114 and a display 116. The display 116 can provide instructions or information to users relating to calibration or therapy. The display 116 can also provide alerts, such an indication of poor connection quality or band not connected. Alerts may also be provided using the feedback generator 114, which can provide haptic feedback to the user, such as upon initiation or termination of stimulation, for reminder alerts, to alert the user of a troubleshooting condition such as connection quality, to perform a tremor inducing activity to measure tremor motion, among others. Accordingly, the user interface components, such as the feedback generator 114 and the display 116 can provide audio, visual, and haptic feedback to the user.

Furthermore, the device 100 can include communications hardware 118 for wireless or wired communication between the device 100 and an external system, such as the user interface device discussed below. The communications hardware 118 can include an antenna. The communications hardware 118 can also include an Ethernet or data bus interface for wired communications.

While the illustrated figure shows several components of the device 100, some of these components are optional and not required in all embodiments of the device 100. In some embodiments, a system can include a diagnostic device or component that does not include neuromodulation functionality. The diagnostic device could be a companion wearable device connected wirelessly through a connected cloud server, and include, for example, sensors such as cardiac activity, skin conductance, and/or motion sensors as described elsewhere herein.

In some embodiments, the device 100 can also be configured to deliver one, two or more of the following: magnetic, vibrational, mechanical, thermal, ultrasonic, or other forms of stimulation instead of, or in addition to electrical stimulation. Such stimulation can be delivered via one, two, or more effectors in contact with, or proximate the skin surface of the patient. However, in some embodiments, the device is configured to only deliver electrical stimulation, and is not configured to deliver one or more of magnetic, vibrational, mechanical, thermal, ultrasonic, or other forms of stimulation.

Although several neurostimulation devices are described herein, in some embodiments nerves are modulated non-invasively to achieve neuro-inhibition. Neuro-inhibition can occur in a variety of ways, including but not limited to hyperpolarizing the neurons to inhibit action potentials and/or depleting neuron ion stores to inhibit firing action potentials. This can occur in some embodiments via, for example, anodal or cathodal stimulation, low frequency stimulation (e.g., less than about 5 Hz in some cases), or continuous or intermediate burst stimulation (e.g., theta burst stimulation). In some embodiments, the wearable devices have at least one implantable portion, which may be temporary or more long term. In many embodiments, the devices are entirely wearable and non-implantable.

### User Interface Device

FIG. 1B illustrates communications between the neurostimulation device 100 and a user interface device 150 over a communication link 130. The communication link 130 can be wired or wireless. The neuromodulation (e.g., neurostimulation) device 100 is capable of communicating and receiving instructions from a user interface device 150. The user interface device 150 can include a computing device. In some embodiments, the user interface device 150 is a mobile computing device, such as a mobile phone, a smartwatch, a tablet, or a wearable computer. The user interface device 150 can also include server computing systems that are remote from the neurostimulation device. The user interface device 150 can include hardware processor(s) 152, a memory 154, display 156, and power electronics 158. In some embodiments, a user interface device 150 can also include one or more sensors, such as sensors described elsewhere herein. Furthermore, in some instances, the user interface device 150 can generate an alert responsive to connection quality between the electrodes and skin of the patient. The alert may be received from the neurostimulation device 100.

In additional embodiments, data acquired from the one or more sensors 102 is processed by a combination of the hardware processor(s) 108 and hardware processor(s) 152. In further embodiments, data collected from one or more sensors 102 is transmitted to the user interface device 150 with little or no processing performed by the hardware processors 108. In some embodiments, the user interface device 150 can include a remote server that processes data and transmits signals back to the device 100 (e.g., via the cloud).

### Controller

FIG. 1C illustrates a block diagram of an embodiment of a controller 160. The controller 160 can include one or more software engines for performing the processes and functions described herein. The software engines can include programmed instructions for performing processes as discussed herein (and illustrated in flowcharts) for detection of input conditions and control of output conditions based on determination of connection quality. The engines can be executed by the one or more hardware processors of the neurostimulation device 100 alone or in combination with the user interface device 150. The programming instructions can be stored in a memory 110. The programming instructions can be implemented in C, C++, JAVA, or any other suitable programming languages. In some embodiments, some or all of the portions of the controller 200 including the engines can be implemented in application specific circuitry such as ASICs and FPGAs. Some aspects of the functionality of the controller 200 can be executed remotely on a server (not shown) over a network. Accordingly, the controller 160 can be implemented with the hardware components described above with respect to FIG. 1A.

### Impedance Determination

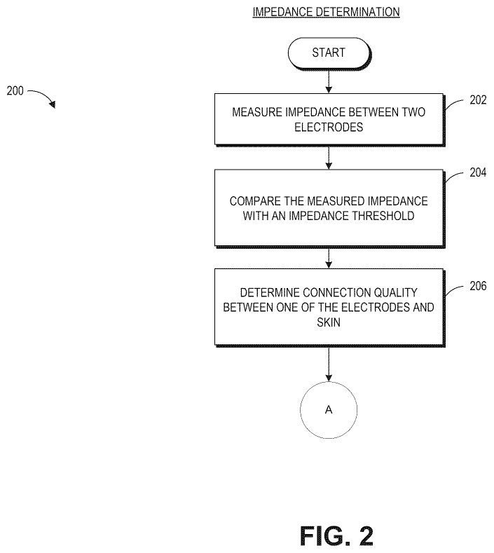

FIG. 2 illustrates a flowchart of an example process 200 for determining connection quality using impedance. The process 200 can be implemented by any of the systems discussed above. The process 200 can be implemented alone or in combination with other processes described below.

The impedance determination process 200 can begin at block 202 with measuring impedance between any two electrodes of the neurostimulation device 100. The determination of impedance can indicate a presence of an open circuit. For example, when there is a poor connection between the electrodes and the skin, the impedance value may be high, corresponding to an open circuit. Such high impedance can result in an improper delivery of electrical stimulation, such as an increase in current density. Accordingly, the impedance measurement can be used to determine connection quality between the electrodes and the skin. In some instances, impedance can be measured for some or all pairs of the electrodes 102.

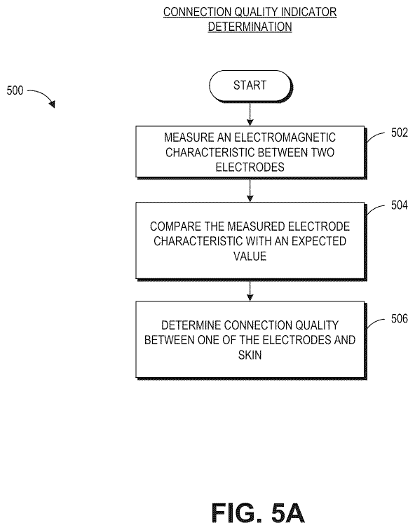

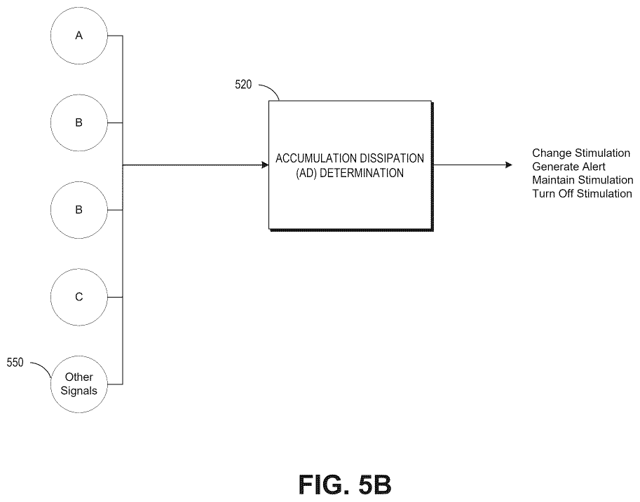

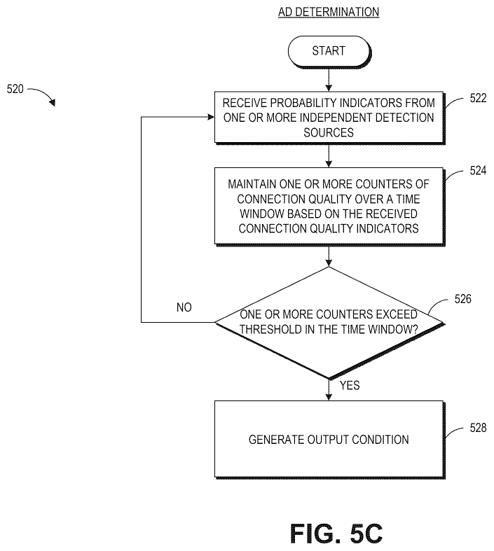

At block 204, the controller 160 can compare the measured impedance with an impedance threshold value. This threshold value might be stored in the memory 110. Based on the comparison, the controller 160 can determine connection quality between the electrodes and the skin of the patient. The impedance for a skin is generally about 4 kΩ. The threshold value can be a factor of the general skin impedance. Example values include about, at least about, or no more than about 8 kΩ, 20 kΩ, 40 kΩ, 50 kΩ, or about 100 kΩ, or ranges including any two of the foregoing values. In some instances, the threshold value is at least about 4 kΩ. If the comparison indicates an open circuit condition, the controller 160 can set connection quality to be low or poor. The connection quality can be a numeric indicator. The numeric indicator can represent a degree of quality of connection. In some instances, the connection quality can be a binary indicator, indicating true (1) or false (0) for whether the connection quality is good or bad. The connection quality can also be a textual indicator, such as “Band Not Connected.” The connection quality indicator can be used by the controller 160 to determine output conditions as discussed in more detail below with respect to FIG. 5. Output conditions can include alerts and/or control of the neurostimulation device 100. For example, output conditions can include halting the stimulation, changing a characteristic of the stimulation, or maintaining a current stimulation treatment. In some instances, the controller 160 can use the impedance detection process 200 by itself to determine the connection quality indicator and control output conditions.

### Pulse Shape Determination

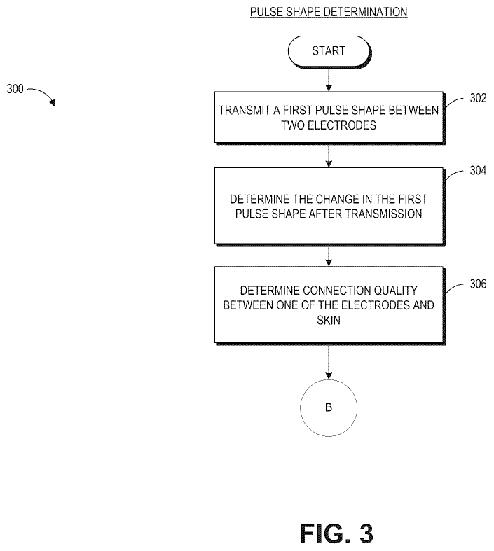

FIG. 3 illustrates a flowchart of an example process 300 for determining connection quality using pulse shape. The process 300 can be implemented by any of the systems discussed above. The process 300 can be implemented alone or in combination with other processes described herein, such as the impedance determination process 200.

The pulse shape determination process 300 can begin at block 302 with a transmission of a pulse having a particular shape across any two electrodes of the neurostimulation device 100. In some instances, the pulse shape can correspond to a positive pulse. In other instances, the pulse shape can correspond to a negative pulse. In other instances, the pulse shape can correspond to a positive and a negative pulse, with an interpulse interval. The pulse shape can also be gaussian or any other profile. The controller 160 can use predetermined pulse shapes stored in the memory 110. The controller 160 can cause the transmission of the pulse at block 302.

When the connection quality is poor, the pulse shape is distorted as it is transmitted across the electrodes. Higher distortion generally corresponds to poor quality connection. Accordingly, the controller 160 can determine the change in the profile of the pulse that was transmitted. In some instances, the change is determined based on the area under the curve of the received pulse. In some instances, the change is determined based on features of the pulse in the time domain, such pulse width or pulse amplitude. The change may also be determined based on a phase of the waveform. Further, the change may also be determined based on duration or time points of wave shape. The change can also be determined based on cross-correlation of the transmitted and received pulse or any other suitable signal processing techniques. In some instances, about or at least about 10%, 15%, 20%, 25%, 30%, or more change in pulse shape or pulse shape features is used as a threshold to determine connection quality indicator. Other values of thresholds can be used to adjust sensitivity.

Based on the change in pulse profile, the controller 160 can determine the connection quality between the electrodes and the skin at block 306. As discussed above, the connection quality can be a numeric, binary, or a textual indicator. The connection quality indicator can be used by the controller 160 to determine output conditions as discussed in more detail below with respect to FIG. 5. In some instances, the controller 160 can use the pulse shape determination process 300 by itself to determine the connection quality indicator and control output conditions.

### Voltage Mismatch Determination

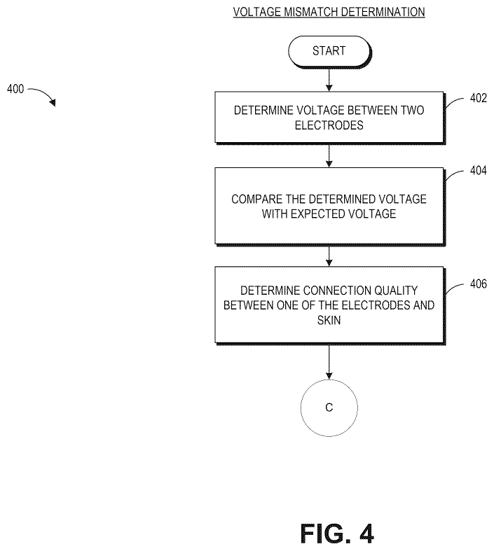

FIG. 4 illustrates a flowchart of an example process 400 for determining connection quality using voltage mismatch. The process 400 can be implemented by any of the systems discussed above. The process 400 can be implemented alone or in combination with other processes described herein, such as the impedance determination process 200 and/or the pulse shape determination process 300.

In several embodiments, the pulse shape determination process 400 can begin at block 402 with a determination of voltage across any two electrodes of the neurostimulation device 100. In some instances, the controller 160 can test the voltage difference across two electrodes. The actual voltage measured can be a function of connection quality between the electrodes and skin. If the connection quality is good, the actual voltage measured will be close to the expected value. In contrast, for a poor quality connection the actual voltage may deviate from the expected value. Accordingly, the controller 160 can compare the measured voltage with the expected voltage at block 404. Example values of thresholds for the voltage difference include about 5k mV, about 10k mV, about 50k mV, about 70k mV, and about 100k mV or more or less, or ranges including any two of the foregoing values.

Based on the comparison, the controller 160 can determine an indication of connection quality between the electrodes and the skin at block 406. In some instances, the controller 160 can use the voltage mismatch determination process 300 by itself to determine the connection quality indicator and control output conditions.

### Connection Quality Indicator Determination

FIG. 5A illustrates an example process 500 for determination of connection quality indicator. The process 500 can be implemented by any of the systems described above. The process 500 can incorporate any of the processes discussed above with respect to FIGS. 2 to 4.

That is, at block 502, the controller 160 can measure an electromagnetic characteristic between two electrodes. The electromagnetic characteristic can include voltage mismatch, impedance determination, or pulse shape determination as discussed above. In some embodiments, the electromagnetic characteristic does not include one or more of voltage mismatch, impedance determination, or pulse shape determination. Other electromagnetic characteristics can include signal quality of delivered current, including but not limited to signal to noise ratio.

At block 504, the controller 160 can compare the measured electrode characteristic with an expected value. For example, the controller 160 can compare the measured values with stored thresholds to identify an open circuit, or pulse shape distortion, or voltage mismatch as discussed above.

Based on the comparison, the controller 160 can determine connection quality indicator between one of the electrodes and skin of the patient at block 506. As discussed above, the connection quality indicator can be a numeric, binary, or a textual indicator. The connection quality indicator can be used by the controller 160 to determine output conditions. Output conditions can include alerts and/or control of the neurostimulation device 100. Alerts can include haptic feedback, visual display, and halting stimulation. For example, output conditions can include halting the stimulation, changing a characteristic of the stimulation, or maintaining current stimulation treatment.

### Accumulation Dissipation (AD) Determination

The controller 160 can use any of the processes 200, 300, or 400 discussed above individually or in combination to determine connection quality and control output conditions. However, changing an output condition based on the determination of the connection quality from one of the processes instantaneously or relative instantaneously can result in false alarms or unnecessarily high sensitivity to connection condition. FIG. 5B illustrates an architecture for combining the processes 200, 300, and 400 and adding a time component (or persistence) with the AD determination 520 (discussed in detail with respect to FIG. 5C) before executing a change the output condition.

The AD determination 520 can use outputs from some or all of the processes described above as shown in FIG. 5B. The outputs may be weighted equally or unequally. In some instances, the AD determination 520 can receive outputs from multiple pulse shape determinations processes 300, each including a different pulse shape. In one example, the AD determination 500 uses a positive pulse and a negative pulse as the transmitted pulses in the pulse shape determinations processes 400. The AD determination 520 can also use other signals 550 to determine output conditions. For example, the accelerometer data from IMU 102 can be used the by controller 160 to determine sudden movements by the user. The controller 160 can correlate the movement data with the connection quality indicator data received from one of the processes discussed above. Other signals 550 can also include user inputs indicating their preference on connection quality threshold and when to change the output conditions.

The output conditions can include, for example, generating an alert, changing neuromodulation (e.g., stimulation) properties, halting the neuromodulation (e.g., stimulation), or maintaining neuromodulation (e.g., stimulation). The time component will be described below with respect to FIG. 5C.

FIG. 5C illustrates a flowchart of an example process 520 for AD determination that adds time component to the processes discussed above. The process 520 can be implemented by any of the systems discussed above.

At block 522, the controller 160 can receive connection quality indicators from the one or more processes 200-400 discussed above. In some examples, as illustrated in block 524, the controller 160 can maintain one or more counters in the memory 110 corresponding to overall connection quality based on the received connection quality indicators from each of the processes shown in FIG. 5A. In one example, a single counter is used by the controller 160 to determine overall connection quality. When one of the processes 200-400 as shown in FIG. 5A indicates a poor connection quality, the controller 160 can increase the counter by 1. If one of the processes outputs a good connection quality indicator, the counter can be decreased by 1. The poor and good are relative terms. As discussed above, the connection quality indicators can be numeric, binary, or textual. While the counters are described in terms of integers and changing in a particular direction, this is relative and other means for tracking can be used.

In some instances, the controller 160 can maintain separate counters for each of the processes illustrated in FIG. 5A. That is, whenever a connections quality indicator is good or bad for a respective process, the respective counter for that process is increased or decreased accordingly by the controller 160. These counters can be stored in the memory 110.

At block 526, the controller 160 can compare the one or more counters to respective one or more threshold. If a single counter is used, the controller 160 compares the single counter with the threshold. When the counter exceeds a threshold, the controller 160 can generate an output condition as shown in block 528. If multiple counters are used, such as a counter for each of the processes input, the controller 160 can compare each of these counters with its respective threshold value. In one example, if any one of the counters exceed their respective threshold, the controller 160 can generate an output condition as shown in block 508. If the counters are within their limits, the controller 160 can continue monitoring the connection quality. In some instances, the controller 160 can poll one or more processes discussed above in a loop at regular intervals. Further, in some examples, the processes can run in parallel and send an indication at regular intervals.

In some instances, the time window is 125 milliseconds, 250 milliseconds, or 500 milliseconds, or more or less. The one or more counters may be initialized to zero at the start of a new time period. The time window can also be continuous. Thus, in some instances, the counters are not initialized after any particular time and maintained throughout the therapy time. Furthermore, in some cases, the threshold count is 64. In other examples, the threshold count can be 128 or higher. In additional examples, the threshold count can be less than 64, between about 32 and 512, or about 32, 64, 128, 256, 512, or ranges including any two of the foregoing values. The users may also be able to set threshold count based on their preferences. Output condition can include changing properties of the stimulation (for example, frequency, voltage, current, duration); halting the stimulation, or generating an alert that can be visual, auditory, or haptic.

Thus, based on the AD determination 520, the controller 160 can adjust or tune when to change the output or operating condition of the neurostimulation device 100 to balance between safety and efficacy. The threshold counts, like threshold values discussed above, can be adjusted to change sensitivity. For example, increasing the threshold count can make the neurostimulation device 100 less sensitive to connection quality errors, while decreasing the threshold count can make the neurostimulation device 100 more sensitive to connection quality errors. In some instances, the threshold counts and values can be set externally by a hospital or manufacturer and can be updated remotely.

### Terminology

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. References to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. The methods disclosed herein include certain actions taken by a practitioner; however, they can also include any third-party instruction of those actions, either expressly or by implication. For example, actions such as “percutaneously stimulating an afferent peripheral nerve” includes “instructing the stimulation of an afferent peripheral nerve.”

1. (canceled)

2. A system for transcutaneously stimulating one or more peripheral nerves of a user, the system comprising:
circuitry for generating stimulation signals;
a plurality of effectors configured to transmit the stimulation signals; and
one or more hardware processors configured to:
measure a plurality of characteristics across at least two of the plurality of effectors,
wherein the at least two of the plurality of effectors comprises a first effector and a second effector,
wherein the plurality of characteristics comprises a first characteristic of the first effector, and
wherein the plurality of characteristics comprises a second characteristic of the second effector;
determine a plurality of connection quality indicators based on the first characteristic and the second characteristic, said connection quality indicators correspond to a degree of connection between the at least two of the plurality of effectors with skin of the user; and
change the stimulation signals based on said degree of connection.

3. The system of claim 2, wherein the one or more hardware processors is further configured to maintain one or more counters for the plurality of connection quality indicators over a time window based on repeated measurements of the plurality of characteristics within the time window;
wherein maintaining one or more counters for the plurality of connection quality indicators comprises determining if at least one of the one or more counters crosses a threshold within the time window; and
change an operating characteristic of the system if at least one of the one or more counters has crossed the threshold within the time window.

4. The system of claim 3, wherein the one or more hardware processors are configured to reset the one or more counters over the time window.

5. The system of claim 3, wherein the one or more hardware processors are configured to maintain stimulation during the time window.

6. The system of claim 3, wherein the operating characteristic includes stimulation settings.

7. The system of claim 3, wherein the changing the operating characteristic includes generating an alert.

8. The system of claim 2, wherein the first characteristic comprises at least on of an impedance and a voltage mismatch.

9. The system of claim 2, wherein the first effector is configured to stimulate one or more of a median nerve and a vagus nerve.

10. The system of claim 2, wherein the first characteristic comprises a pulse shape determination.

11. The system of claim 2, wherein each of the plurality of characteristics is configured to enable an independent determination of the connection quality indicator independent of at least one other determination of the connection quality indicator.

12. A system for transcutaneously stimulating one or more peripheral nerves of a user, the system comprising:
circuitry for generating stimulation signals;
a plurality of effectors configured to transmit the stimulation signals; and
one or more hardware processors configured to:
measure a plurality of characteristics across at least two of the plurality of effectors,
determine a plurality of connection quality indicators based on the plurality of characteristics,
wherein said connection quality indicators correspond to a degree of connection between the at least two of the plurality of effectors with skin of the user; and
change an operating characteristic of the system when at least one of the plurality of connection quality indicators exceeds a threshold.

13. The system of claim 12,
wherein the at least two of the plurality of effectors comprises a first effector and a second effector,
wherein the plurality of characteristics comprises a first characteristic of the first effector, and
wherein the plurality of characteristics comprises a second characteristic of the second effector.

14. The system of claim 12, wherein characteristic comprises a voltage mismatch or an impedance.

15. The system of claim 12, wherein characteristic comprises a pulse shape determination.

16. The system of claim 12, wherein the operating characteristic includes stimulation settings.

17. The system of claim 12, wherein the changing the operating characteristic includes generating an alert.

18. The system of claim 12, wherein the one or more hardware processors is configured to determine a plurality of characteristics across the plurality of effectors, wherein each of the plurality of characteristics is configured to enable an independent determination of the connection quality indicator.

19. The system of claim 18, wherein the one or more hardware processors is configured to determine a plurality of connection quality indicators based on the determined plurality of characteristics.

20. The system of claim 19, wherein the one or more hardware processors is configured to track the determined plurality of connection quality indicators over a time period.

21. The system of claim 20, wherein the operating characteristic is only changed when at least one of the determined plurality of connection quality indicators exceed a threshold over the time period.
